(19) United States
(12) Patent Application Publication
(10) Pub. No.: US 2004/0009194 A1
(43) Pub. Date: Jan. 15, 2004

(54) Methods, and compositions for a therapeutic antigen presenting cell vaccine for treatment of immunodeficiency virus

(76) Inventors: Jean-Marie Andrieu, Paris (FR); Louis Lu, Paris (FR)

Correspondence Address: IP DEPARTMENT OF PIPER RUDNICK LLP, 3400 TWO LOGAN SQUARE, 18TH AND ARCH STREETS, PHILADELPHIA, PA, 19103, US

(21) Appl. No.: 10/600,361

(22) Filed: Jun. 20, 2003

Related U.S. Application Data
(60) Provisional application No. 60/390,625, filed on Jun. 21, 2002.

(51) Int. Cl. A61K 39/21; A61K 31/551

(52) U.S. Cl. 424/208.1; 514/220

(57) ABSTRACT

One aspect of this invention provides a composition capable of eliciting an immune response to an immunodeficiency virus in mammals, wherein the composition is comprised of an inactivated virus-pulsed antigen presenting cell. In another aspect the aforementioned composition may also contain a combination of an inactivated virus-pulsed antigen presenting cell and an immunodeficiency protease inhibitor. Still other other aspects of this invention provide for methods of treating mammals with an inactivated virus-pulsed antigen presenting cell, the vaccines related to such cells.

### CROSS REFERENCE TO RELATED APPLICATION

This application claims the benefit of U.S. Provisional Application No. 60/390,625 filed Jun. 21, 2002, the subject matter of which is fully incorporated herein by reference.

### FIELD OF THE INVENTION

Aspects of this invention relate to methods and compositions for treating immunodeficiency viruses. More specifically, selected aspects of this invention are related to inactivated-virus pulsed antigen presenting cells capable of eliciting an immune response against immunodeficiency viruses.

### BACKGROUND

Although introduction of highly active antiretroviral therapy (HAART) including at least one Human Immunodeficiency Virus (HIV) protease inhibitor (PI) allows dramatic decreases in plasma HIV RNA loads and significant recovery of the T-cell compartment in the majority of patients, HIV eradication by prolonged HAART treatment appears to be unlikely due to the persistence of a cellular reservoir of infectious HIV. On the other hand, HAART-treated patients fail to mount anti-HIV immunity, as evidenced by either, the rapid viral rebound observed in almost all patients after discontinuation of HAART, or by a maintained high viral load in patients experiencing a virologic failure despite significant T-cell recovery. Recent studies have demonstrated that the lack of functional virus-specific effector T lymphocytes is a key immunologic feature of chronic HIV and Simian Immunodeficiency Virus (SIV) infections.

Antigen-presenting cells are important for initiating and maintaining virus-specific immunity. An effective cell-mediated immune response against human immunodeficiency virus (HIV) or simian immunodeficiency virus (SIV) is known to be one type of antigen-presenting cell called a dendritic cell that is critical to achieve the control of viral replication. Recent studies have shown that (DC) cultured from the peripheral blood of HIV-negative donors or SIV-negative animals and pulsed with chemically (2,2′-dithiodipyridine or aldrithiol-2) inactivated HIV or SIV are potent stimulators of primary MHC class-I-restricted T-cell responses in vitro.

Despite significant immune recovery with potent highly active antiretroviral therapy (HAART), eradication of human immunodeficiency virus (HIV) from the bodies of infected individuals represents a challenge. For purposes of further clarification we have provided herein a list of relevant terms and their general meaning as is understood within the art. These definitions are included to provide a general background into the terminology used throughout the specification and in no way are intended as a limitation to the breadth of the invention which is defined in the appended claims.

Antiretroviral therapy usually is related to a substance that stops or suppresses the activity of a retrovirus such as HIV. AZT, ddC, ddI and d4T are examples of antiretroviral drugs.

AZT, which is just one example of a nucleoside analogue, is used to slow replication of HIV. More specifically, AZT is approved for the initial treatment of HIV infection. AZT is increasingly administered in combination with other antiviral drugs, especially 3TC (a combination that is under consideration by the FDA as another initial treatment regimen for HIV) as well as ddC (an FDA-approved combination for persons with progressive disease and CD4 cell counts below 300).

DDC, is another exemplary nucleoside analogue that inhibits infection of new cells by HIV. It is FDA-approved for the treatment of HIV when used in combination with AZT in patients with CD4 cell counts below 300 who have deteriorated despite treatment and as monotherapy following AZT-failure.

Antigen-Presenting Cells (APC), are generally known in the art as immunocompetent cells, usually positive, that mediate the cellular immune response by processing and presenting antigens or mitogens, which stimulate T-cell activation. An exemplary antigen presenting cell is a cell that carries on its surface antigen bound to MCH Class I or Class II molecules and presents the antigen in this context to T-cells. This includes, for example, macrophages, endothelium, langerhans cells of the skin. Another example of an antigen-presenting cell is the monocyte-derived dendritic cells (DC), which is believed to be the most potent APC capable of priming major histocompatibility complex class I- and II-restricted antigen-specific T-cell responses.

Antigens are substances which are capable, under appropriate conditions, of inducing a specific immune response and of reacting with the products of that response, that is, with specific antibodies or specifically sensitized T-lymphocytes, or both. Antigens may be soluble substances, such as toxins and foreign proteins, or particulate, such as bacteria and tissue cells. However, only the portion of the protein or polysaccharide molecule known as the antigenic determinant (epitopes) combines with antibody or a specific receptor on a lymphocyte. An example of an antigen is a virus coded cell surface antigens that appear soon after the infection of a cell by virus, but before virus replication has begun.

It should be understood that the phrase “virus-specific cell” as it is used herein, and generally understood in the art, describes a cell that it is specifically configured to attack a particular virus

Effector cells, are generally known and understood as a terminally differentiated leukocyte that performs one or more specific functions.

CD4 is a 55-kD glycoprotein originally defined as a differentiation antigen on T-lymphocytes, but also found on other cells including monocytes/macrophages. CD4 antigens are members of the immunoglobulin supergene family and are implicated as associative recognition elements in MHC (major histocompatibility complex) class II-restricted immune responses. On T-lymphocytes they define the helper/inducer subset. CD4 antigens also serve as HIV receptors, binding directly to the envelope protein gp120 on HIV.

CD 4 receptors are an example of the protein structure on the surface of a human cell that allows HIV to attach, enter, and thus infect a cell. CD4 receptors are present on CD4 cells (helper T-cells), macrophages and dendritic cells, among others. Normally, CD4 acts as an accessory molecule, forming part of larger structures (such as the T-cell receptor) through which T cells and other cells signal each other.

CD8 are differentiation antigens found on thymocytes and on cytotoxic and suppressor T-lymphocytes. CD8 antigens are members of the immunoglobulin supergene family and are associative recognition elements in major histocompatibility complex class I-restricted interactions. CD8-+T-lymphocytes are a critical subpopulation of regulatory T-lymphocytes involved in MHC class I-restricted interactions. They include both cytotoxic T-lymphocytes (T-lymphocytes, cytotoxic) and suppressor T-lymphocytes (T-lymphocytes, suppressor-effector).

A CD8 cell is one type of T-lymphocyte which bears the CD8 molecular marker on its surface. Some CD8 cells recognize and kill cancerous cells and those infected by intracellular pathogens (some bacteria, viruses and mycoplasma). These cells are called cytotoxic T-lymphocytes (see). An example of such type of CD8 cell is the CD8+T cell.

A cytotoxic t-lymphocyte is a type of CD8 or, CD4 lymphocyte that kills diseased cells infected by a specific virus or other intracellular microbe. CTLs interact with Major Histocompatibility Complex (MHC) class I receptors.

A T-cell is a class of lymphocytes, so called because they are derived from the thymus and have been through thymic processing. They are involved primarily in controlling cell-mediated immune reactions and in the control of B-cell development. The T-cells coordinate the immune system by secreting lymphokine hormones. There are 3 fundamentally different types of t cells: helper, killer, and suppressor. Each has many subdivisions. T-cells are also called t lymphocytes. They bear T-cell antigen receptors (CD3) and lack Fc or C3b receptors. Major T-cell subsets are CD4 (mainly helper cells) and CD8 (mostly cytotoxic or suppressor T-cells).

A macaque is any one of several species of short-tailed monkeys of the genus Macacus; as, M. Maurus, the moor macaque of the East Indies.

Immune deficiency diseases are those diseases in which immune reactions are suppressed or reduced. Reasons may include congenital absence of B and/or T lymphocytes or viral killing of helper lymphocytes (see HIV).

Human Immunodeficiency Virus (HIV) is a type of retrovirus that is responsible for the fatal illness, acquired immunodeficiency syndrome (AIDS). Two strains have been identified. Type 1: the retrovirus recognized as the agent that induces AIDS. Type 2: a virus closely related to HIV-1 that also leads to immune suppression. HIV-2 is not as virulent as HIV-1 and is epidemic only in West Africa. Simian Immunodeficiency Virus (SIV), a species of the genus lentivirus, subgenus primate is an immunodeficiency viruses (immunodeficiency viruses, primate), that induces acquired immunodeficiency syndrome in monkeys and apes (SAIDS). One skilled in the art is aware that the genetic organization of SIV is virtually identical to HIV. Siv is 50% homologous in nucleotide sequence to HIV-1. SIV and HIV-2 exhibit close structural and immunologic properties and are 75% homologous. SIV does not cause immune deficiency in its natural host, the African green monkey, but does produce SAIDS in the rhesus macaque. Subgroups of SIV include SIV-1 and SIV-2.

Humoral antibodies (humoral antibody) are antibodies which are secreted by B lymphocytes circulating in the blood, in response to antigens found in body fluids.

All references contained herein, including those references listed in the reference section of this specification, are hereby fully incorporated by reference.

### SUMMARY OF THE INVENTION

This invention relates to the development of a composition capable of suppressing HIV, and other related immunodeficiency virus, such as for example, SIV, HIV-1 and HIV-2.

In one aspect, the invention APCs, which are capable of priming major histocompatibility complex class I- and II-restricted antigen-specific T-cell responses are pulsed with inactivated virus to elicit the expansion of virus specific cells that are capable of killing and or repressing replication of HIV infected cells.

Another aspect of this invention provides for an enhanced expansion of virus specific cells which are capable of killing HIV infected cells and or suppressing HIV replication, combined with inhibitors and inactivated-virus pulsed APCs and subsequently subjecting HIV infected cells to this combined vaccine.

In yet another aspect of this invention, the in vivo treatment with inactivated virus-pulsed APCs from a mammal is provided.

### BRIEF DESCRIPTION OF THE DRAWINGS

FIG. 1 is a table showing characteristics of 30 HIV-1-infected adults (10 who were naive for antiviral treatment, 10 HAART-treated virologic responders, and 10 HAART-treated virologic nonresponders)

FIG. 2 is a graphical representation of the proliferation of patient T cells following stimulation with inactivated-virus-pulsed autologous DC in the absence or presence of HIV PI (indinavir, 10 nM).

FIG. 2A shows the mean (±SD) [³H]thymidine incorporation in T cells from untreated patients in the absence ([□]) or presence (▪) of PI, in T cells from HAART-treated plasma viral load responders in the absence (Δ) or presence (▴) of PI, and in T cells from plasma viral load nonresponders in the absence (◯) or presence (●) of PI.

FIG. 2B shows the Mean (±SD) relative CD4/CD8 ratio in T cells from untreated patients (□), plasma viral load responders (Z,1), and plasma viral load nonresponders (Z,2). The baseline CD4/CD8 ratio in the absence of stimulation was normalized to 1. The baseline ranges of the CD3+ CD4+ phenotype in untreated patients, plasma viral load responders, and plasma viral load nonresponders were 15 to 32%, 24 to 53%, and 21 to 41%, respectively.

FIG. 3 is a pair of graphs showing HIV-1 gag-specific CTL activity in patient T cells expanded by inactivated-virus-pulsed autologous DC in the absence or presence of indinavir.

FIG. 3A illustrates the mean (±SD) percent specific lysis (at an effector/target ratio of 10:1) of autologous B-LCL targets (infected with recombinant vaccinia virus containing a HIV-1 gag gene) by T cells stimulated with virus-pulsed DC with or without PI. T cells were from untreated patients (□), plasma viral load responders to HAART (Z,1), and plasma viral load nonresponders to HAART (Z,2).

FIG. 3B shows the mean (±SD) percent specific lysis from all patients in the absence of antibodies (Z,5) or in the presence of blocking antibodies against CD4 (Z,6) or CD8 (▪). The background percent gag-specific lysis using unloaded DC treated T cells was <10%.

FIG. 4 is a series of graphs showing Quantitative analysis of anti-HIV activity of patient T cells stimulated with inactivated-virus-pulsed autologous DC in the absence or presence of PI. Each result is the mean (±SD) number of proviral HIV DNA copies/10⁶ cells (A and C) or the mean (±SD) number of supernatant HIV RNA copies per milliliter (B and D) in the coculture of autologous virus-pulsed-DC-stimulated T cells and superinfected T cells from untreated patients (□), plasma viral load responders to HAART (Z,1), and plasma viral load nonresponders to HAART (Z,2) or from all patients in the absence of antibodies (Z,5) or the presence of blocking antibodies against CD4 (Z,6) or CD8 (▪).

FIG. 5 is a series of graphs showing functions of DC following treatment with activated-T-cell supernatant.

FIG. 5A shows the mean (±SD) [³H]thymidine incorporation in patient T cells stimulated with virus-pulsed DC (◯) or DC pretreated with activated-T-cell supernatant before (▴) or after (▪) pulsing with inactivated autologous virus.

FIG. 5B shows the mean (±SD) percent HIV gag-specific lysis (at an effector/target ratio of 10:1) of autologous B-LCL targets by patient T cells expanded with virus-pulsed DC (□) or DC pretreated with activated-T-cell supernatant before (Z,1) or after (Z,2) pulsing with inactivated autologous virus.

FIG. 5C shows the mean (±SD) number of proviral HIV DNA copies/10⁶ cells or supernatant HIV RNA copies per milliliter in the coculture of superinfected T cells with autologous T cells expanded with virus-pulsed DC (□) or DC pretreated with activated-T-cell supernatant before (Z,1) or after (Z,2) pulsing with inactivated autologous virus.

FIG. 6 is a series of photographs showing the morphology and phenotype of macaque monocyte-derived DCs.

FIGS. 6A and B are representative of examples of macaque DC morphology (magnification×400).

FIG. 6A shows and unloaded (control) DCs.

FIG. 6B shows AT-2-inactivated SIVmac251-loaded DCs.

FIGS. 6C and D show CD83 expression of macaque DCs (dark red line, monoclonal antibody isotype control; light green line, CD83 labeling).

FIG. 6C further shows unloaded DCs.

FIG. 6D shows AT-2-inactivated SIVmac251-loaded DCs.

FIG. 7 is a series of graphs showing the virologic and immunologic monitoring in immunized and non-immunized macaques. a, PBMC SIV DNA per million cells (geometric mean±s.e.m) in immunized (A) and non-immunized (A) macaques. b, Plasma SIV RNA (geometric mean±s.e.m.) in immunized (▪) and non-immunized (□) macaques. c, Plasma SIV RNA concentrations in immunized animals (▪,monkey no. 1; ▴, no. 2; no. 3; ⋄no. 4; ●, no. 5; □, no. 6; Δ, no. 7; ∇, no. 8;⋄, monkey no. 9; ∘and, 10) and non-immunized animals (monkey no. 11; +, no. 12; *, no. 13; and ▪, no. 14). d, CD4+ count (mean±s.e.m.) in immunized (●) and non-immunized (◯) macaques. e, Neutralizing antibody (NAb) titers (mean±s.e.m.) in immunized (▾) and non-immunized (∇) macaques. f, SIV-specific spot-forming cells (SFCs) frequency (mean±s.e.m.) in immunized (♦) and non-immunized (⋄) macaques. Vertical dotted lines indicate the time points of immunization. *P value <0.05; **P value <0.01.

FIG. 8 is a series of graphs showing the SIV-specific CTL and anti-SIV activity of peripheral T cells.

FIG. 8A and B show SIV-specific cytolysis of AT-2-inactivated SIVmac251-pulsed DCs by PBLs taken at week 6 from immunized animals (monkeys 1-10) and non-immunized animals (monkeys 11-14).

FIG. 8A shows SIV-specific cytolysis at different E:T ratios (mean of triplicate wells; ▪, monkey no. 1; ▴, no. 2; ▾, no. 3; ♦, no. 4; ●, no. 5; □, no. 6; Δ, no. 7; ∇, no. 8; ⋄, no. 9; ◯,no. 10; x no. 11;+, no. 12; *, no. 13; and ▪, no. 14).

FIG. 8B shows SIV-specific cytolysis at a E:T ratio of 40:1 in the absence or the presence of monoclonal antibodies against CD4 or CD8 (Ab) (mean±s.e.m.). CTL assay was controlled by non-pulsed autologous DCs as negative control targets and by recombinant HCV-core-protein-pulsed autologous DCs as non-specific control targets. The background percent of lysis with non-pulsed or control antigen-pulsed autologous DCs was <12%.

FIG. 8C shows cell-associated SIV DNA and supernatant SIV RNA in the absence (open column) or the presence (filled column) of peripheral CD8+T cells taken at week 8 from each group of animals (geometric mean±s.e.m.).

FIG. 9 is a pair of photographs showing the histopathology of macaque LN. a and b, Histological features of follicular dendritic cell network (FDCN) and germinal centers (GC) (H&E staining) in LN examined at week 42 (top, ×25 magnification; bottom, ×200 magnification).

FIG. 9A shows representative features of destruction of FDCN and disappearance of GC from control monkeys (no. 14).

FIG. 9B shows representative features of well-preserved FDCN and GC in LN from vaccinated monkeys (no. 6).

FIG. 10 is a pair of graphs illustrating the virologic and immunologic analysis in macaque LN.

FIG. 10A shows cell-associated SIV DNA or RNA burden (horizontal lines, geometric means) measured in the 20 LN samples of the 10 immunized animals and the 8 LN of the 4 non-immunized animals.

FIG. 10B shows the correlation between cellular SIV DNA (▴) or RNA (♦) and SIV-specific SFCs detected in the 10 immunized animals.

FIG. 11 shows a table of the animal characteristics and immunization schedule.

### DETAILED DESCRIPTION OF THE INVENTION

In view of the current teachings, we have performed experiments and describe herein, that the persistent failure in mounting anti-HIV immunity in untreated or HAART-treated patients is linked to an inadequate or inappropriate signal in virus-specific antigen presentation, possibly resulting from a disturbance in the generation and/or function of antigen-presenting cells (APCs) in chronically immune-activated lymphoid organs or tissues of HIV-infected patients.

One aspect of this invention provides monocyte-derived dendritic cells (DC), which are understood in the art to be one of the most potent APCs capable of priming major histocompatibility complex class I- and II-restricted antigen-specific T-cell responses, pulsed with inactivated autologous virus can result in the expansion of virus-specific CD8+T cells. These virus specific CD8+T cells are capable of killing HIV-infected cells and suppressing HIV type 1 (HIV-1) replication. In yet another aspect of our invention, we have discovered that the combination of Protease Inhibitors (PIs) and inactivated-virus pulsed DC creates a significant expansion of virus specific effector cells. Indeed, a combination of inactivated-virus-pulsed DC and the HIV PI indinavir (at a nonantiviral concentration) resulted in an ample expansion of virus-specific CD8+T cells which was sufficient to eradicate HIV-1 in peripheral blood mononuclear cells (PBMC) taken from HIV infected patients.

In yet another aspect of the invention we discovered the in vivo effect of inactivated virus-pulsed DCs as a vaccine. Our in vivo results demonstrate that a therapeutic vaccine made of inactivated-virus-pulsed APC can elicit effective cellular immune responses against immunodeficiency disease. Still a further aspect provides for allowing the control of virus replication in the secondary lymphoid tissues and the reduction of cell-associated viral DNA and cell-free viral RNA in blood of virus infected mammals.

Reference will now be made in detail to selected preferred embodiments of the invention, which, together with the following examples, serve to explain principles of the invention. The following examples are not intended as a limitation of the Applicants' invention.

Through non-limiting examples this invention illustrates both the efficacy of in vitro and in vivo immune response to two separate immunodeficiency viruses, through the use of inactivated virus pulsed dendritic cells. Namely, these particular examples provide in one aspect of this invention that the DCs of HIV infected patients loaded with AT-2-inactivated autologous HIV, will elicit functional virus-specific effector CD8+T lymphocytes, which are capable of eradicating HIV-infected cells in vitro. It should be understood that any APC capable of being loaded with inactivated virus that is functional to elicit CD8+T is suitable for use in this invention.

Another example demonstrates another aspect of this invention. Namely, the example illustrates that in SIV-infected rhesus monkeys an effective and durable SIV-specific cellular immunity is elicited by a vaccination with chemically inactivated SIV-pulsed dendritic cells (DCs). After three immunizations made at two-week intervals, the animals exhibited a 50-fold decrease of SIV DNA and a 1,000-fold decrease of SIV RNA in peripheral blood. Such reduced viral load levels were maintained over the remaining thirty-four weeks of the study. Molecular and cellular analysis of axillary and inguinal nodes lymphocytes of vaccinated monkeys revealed the direct correlation between decreased SIV DNA and RNA levels and increased SIV-specific T cells responses. Inactivated whole virus-pulsed DC vaccines may be used to control immunodeficiency viruses diseases.

The aforementioned non-limiting examples will now be described in detail. Animals

Twenty colony-bred rhesus macaques (Macaca mulatta) were obtained from Shunde Experimental Animal Center (Guangdong, China). All animals were in good health, 2-4 years old, weighed 4-6 kg and were seronegative for SIV, SRV, simian T cells lymphotropic virus 1, and hepatitis B virus. Macaques were inoculated intravenously with 5 MID100 of pathogenic SIVmac251 (gift of Dr. P. A. Marx from Aaron Diamond AIDS Research Center, New York, USA).

Vaccines

Only the infected animals with plasma SIV loads ranging from 105 to 106 copies/mil (i.e. less than 10 folds difference) were included. Peripheral blood mononuclear cells (PBMCS) were isolated from 20 ml fresh EDTA-treated whole blood using Ficoll-Hypaque density gradient centrifugation. After three washes with Hank's balanced salt solution (Hank's buffer), PBMCs were suspended in 5×10 6/ml of RPMI 1640 medium (Eurobio, Les Ulis, France) containing 0.5% of bovine, serum albumin (Sigma, St Louis, Mo., USA) and then subjected to plastic adherence at a density of 106 cells/m2. After 2-hour incubation at 37° C. in 5% CO2, non-adherent cells were removed by rinsing 3 times with Hank's buffer. The plastic-adherent cells were then cultured for 5 days in AIM-V medium (Life Technologies, Grand Island, N.Y., USA) supplemented with 2000 U/ml GM-CSF (Schering-Plough, Brinny, Ireland) and 50 ng/ml IL-4 (R&D system, Minneapolis, Minn., USA). Following this culture period, non-adherent cell were >90% immature DCs based on their morphology and phenotype (CD 11c+CD 14·).

The SIVmac251 was inactivated by 250 μM aldrithiol (AT)-2 (Sigma) as previously described by Rossio, J. L. et al., J. Virol. 72, 7992-8001 (1998). AT-2-inactivated viruses (109 viral particles/ml) were added to DCs for 2 hours at 37° C. and were then cultured in the AIM-V medium containing 2,000 U/ml GM-CSF, 50 ng/ml IL-4, and 50 ng/ml TNF-α (R&D System) for 2 more days. Thus, DCs were differentiated into a partial mature morphology and phenotype (increasing expression of CD40, CD80, CD83, CD86, and HLA-DR). After three washes with Hank's buffer, inactivated-virus-pulsed autologous DCs were re-suspended in RPMI 1640 culture medium (106/ml). They were then ready for injection to the macaques. Animals received subcutaneous injections of 1 ml (i.e. 0.25 ml in 4 sitcs in close proximity to left and right axillary and ingliinal lymph nodes) with the SIV-pulsed autologous DC vaccine (group A) or the -unloaded autologous DCs as control (group B). Booster injections were given to animals of both groups every 2 weeks for 8 weeks.

Viral-Load Measurements

SIV RNA in plasma and supernatants was quantified by a previously described quantitative assay 16 with a detection threshold of 10 copies/ml using primers (sense, 5′-ATGTAGTATGGGCAGCAAATGAAT-3′,antisense,5′GTGCTGTTGGTCTACTTGTTTTTG-3′) (SEQ ID No. 1) and probe (5′-GATTTGGATTAGCAGAAAGCCTGT TGGAGAACAAAGAA-3′) (SEQ ID No. 2), specifically optimized for SIVmac251. Cell-associated SIV DNA and RNA were quantified as previously described in Example 1 by -using the above SIVmac251-specific primers and probe.

Flow Cytometry

CD4+ and CD8+T-cell counts (CD3+CD4+ and CD3+CD8+) and DCs (CD14−, CD11c+, CD40+, CD80+, CD83+, CD86+, and HLA-DR+) were assessed by flow-cytometry analysis (FACScan, Beckton Dickinson [BD], San Jose, Calif., USA) using a panel of direct fluorescence-labeled monoclonal antibodies validated for the rhesus macaque study: anti-CD3-FITC (clone SP34), CD4-PE (clone MT477), CD8-PcrCP (clone RPA-T8), CD14-PE (clone M5E2), CD40-PE (clone 5C3), CD80-PE (clone L307.4), CD83-PE (cloncHB15e), CD86-PE (clone 2331 [FUN-1.]), and HLA-DR-PE (clone G46-6) (BD Biosciences) and CD11c-FITC (clone 3.9) (sigma).

ELISPOT Assay

The IFN-γ ELISPOT assay was performed in uncultured PBLs or lymph node cells (2×105) using a rhesus-macaque-specific commercial kit (Autoimmun Diagnostika [AID] GmbH, Straβberg, Germany). AT-2-inactivated SIV-pulsed autologous DCs (2×104) were used as virus-specific antigen stimulators. The data were read with an automated ELISPOT reader (AID). The number of SIV-specific spot forming cells (SFCs) were calculated by subtracting the nonspecific SPCs in the presence of unpulsed autologous DCs with the use of a build-in software (Elispot 2.9, AID),

CTL Assay

The SIV-specific CTL assay was performed in uncultured PBLs or lymph node cells using AT-2-inactivated SIV-pulsed autologous DCs as target cells as previously described 17. The percentage of specific cytolysis was calculated by subtracting the nonspecific 51Cr release of the wells in the presence of recombinant hepatitis C virus core protein (a gift of Dr. D. Han from Chinese Academy of Medical Sciences, Beijing, China)-pulsed autologous DCs.

Antiviral Activity Assays

The anti-STV activity was assessed in peripheral or lymph node CD8+T cells using a previously described method 18. The same SIVmac251 (100 50% tissue culture infective dose) was used to superinfect macaque's CD4+T cells as targets. Cell associated SIV DNA and supernatant RNA was monitored by quantitative PCR and RT-PCR (see above).

Statistical Analysis

Impaired data between different groups of animals or paired data before and after immunization were compared by the Mann-Whitney or the Wilcoxon test respectively.

### EXAMPLE 1

One aspect of this example illustrate the proliferation of patient T cells following stimulation with virus-pulsed autologous DC.

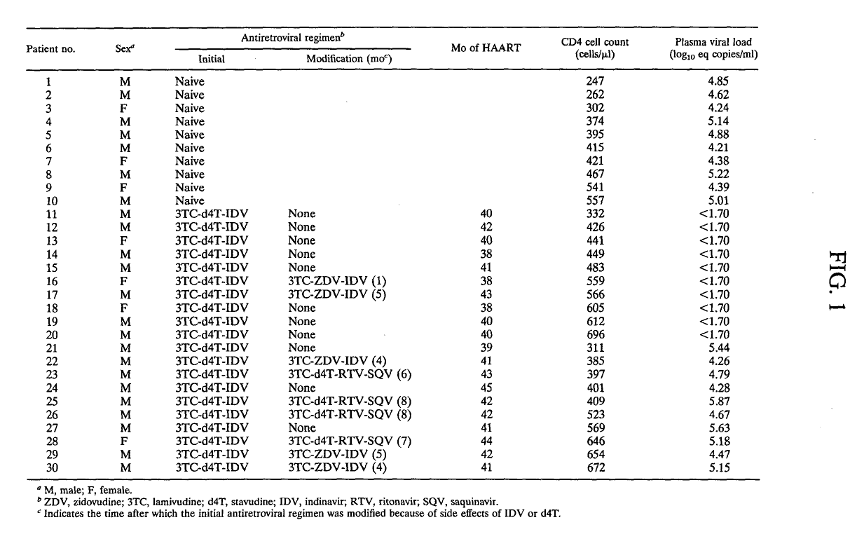

Viruses were isolated from 10 untreated asymptomatic HIV-seropositive patients (CD4 cell count, 200 to 600 cells/μl; plasma HIV RNA load, 4 to 6 log¹⁰ eq copies/ml) and 20 patients treated by prolonged HAART (>3 years) (CD4 cell count, 300 to 700 cells/μl; 10 patients with virologic response [plasma HIV RNA load, <50 log¹⁰ eq copies/ml] and 10 patients with virologic resistance [plasma HIV RNA load, 4 to 6 log¹⁰ eq copies/ml]). (FIG. 1). As will be well understood in the art, the use of autologous cells is beneficial for a number of reasons, including the cognitive response of the patient, but it should be understood that heterologous cells may be used to practice this invention.

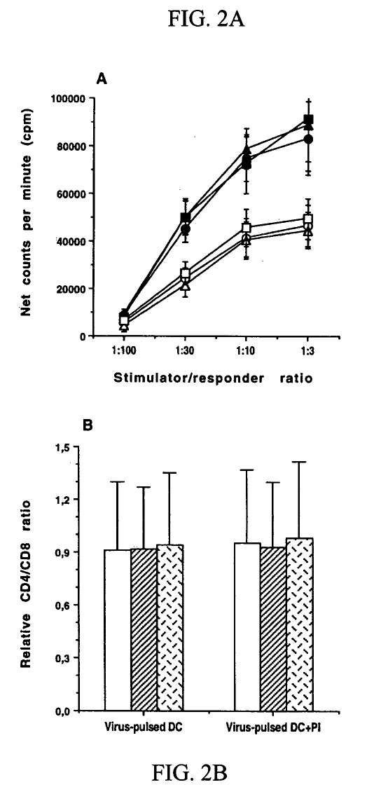

To mimic the antigen capture by DC in peripheral tissues, immature DC (i.e., competent in antigen capture) generated by culturing patient blood monocytes with GM-CSF and IL-4 for 5 days were pulsed with autologous viral isolates inactivated by 2,2′ dithiodipyridine or aldrithiol-2 (AT-2), which preserves the intact native conformation and fusogenic activity of HIV Env protein. As will be appreciated by those skilled in the art, there are a number of mechanisms suitable to inactivate a virus, and any of these methods can be used so long as the method preserves the intact native conformation and fusogenic activity of HIV Env protein. To model the presentation of antigens in lymphoid tissue, virus-pulsed DC were matured in the presence of TNF-α and IFN-α for an additional 3 days to maximize their T-cell-stimulatory activity. Matured virus-pulsed DC was then used to stimulate autologous PBL at a stimulator/responder ratio of 1:3 in the absence or presence of PI at a nonantiviral concentration (10 nM indinavir). By day 7 of coculture, PBL were restimulated with the same virus-pulsed DC for an additional 7 days. At day 14, proliferation was measured by incubating PBL with virus-pulsed DC at stimulator/responder ratios of 1:3 to 1:100. Inactivated virus-pulsed DC stimulated [methyl-3H] thymidine incorporation by autologous PBL from both untreated and HAART-treated patients independently of their blood CD4 cell counts and plasma viral loads (P>0.5), while this DC-mediated T-cell proliferation was significantly enhanced by the presence of PI (at a nonantiviral dose) (P<0.001) (FIG. 2A). Phenotype analysis by flow cytometry showed that both CD4+ and CD8+T cells were equally stimulated by virus pulsed DC in the presence or absence of PI (FIG. 2B). HIV-1 gag-specific CTL activity following stimulation with virus-pulsed autologous DC.

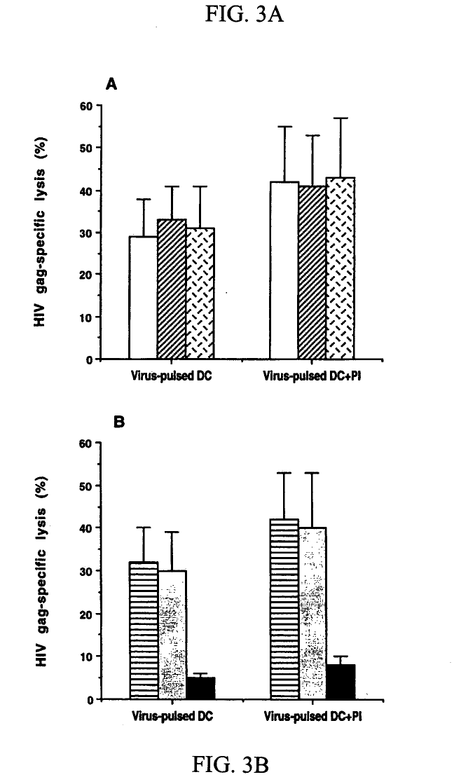

Another aspect of this example evaluated HW-specific cytotoxic-T-lymphocyte (CTL) activity of autologous PBL expanded with inactivated-virus-pulsed DC using as target cells autologous B-LCL infected by recombinant vaccinia virus containing a gag gene of HIV-1 as described previously. HIV gag-specific B-LCL killing was up-regulated by autologous PBL stimulated with virus-pulsed DC (at an effector/target ratio of 10:1) in both untreated and HAART-treated patients independently of their blood CD4 cell counts and plasma viral loads (P>0.4). Such a gag-specific CTL activity was significantly enhanced (P<0.01) by the presence of indinavir (10 nM) (FIG. 3A). Similar enhancement by PI was also observed with PBL stimulated with virus-pulsed DC alone when the effector/target ratio was increased to 50:1. This CTL-mediated B-LCL killing was executed exclusively by CD8+T cells, since cell killing was blocked by the addition of anti-CD8 antibodies whereas it was unaffected by the addition of anti-CD4 antibodies (FIG. 3B). Anti-HIV activity of patient T cells following stimulation with virus-pulsed autologous DC. Having observed that virus pulsed DC were capable of stimulating the proliferation and CTL activity of autologous T cells from both untreated and HAART-treated patients regardless of their CD4 cell count and level of HIV viremia, we then examined the direct antiviral activity of autologous T cells expanded by virus-pulsed DC in the presence or the absence of PI. To minimize the variation in the frequency of PBMC harboring infectious HIV among untreated and HAART-treated patients, all patient PBMC were superinfected with the same dose (100 50% tissue culture infective doses) of autologous isolates as described previously.

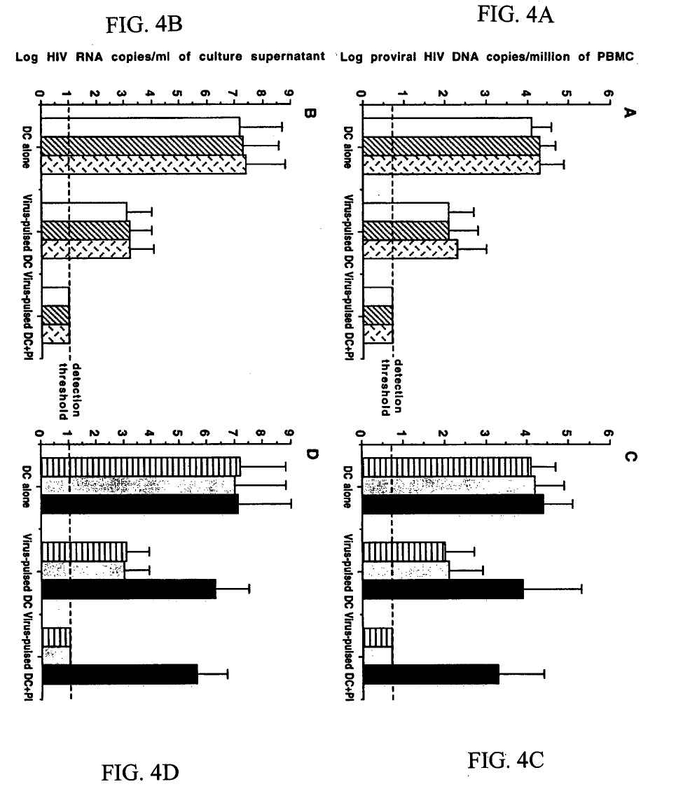

The total HIV proviral DNA concentrations (means±standard deviations [SDs]) measured before and after 12 h of superinfection were 2.9±0.5 (range, 2.1 to 3.8) and 4.1 ±0.2 (range, 3.7 to 4.5) log₁₀ copies per million PBMC, respectively. To mimic immune activation in lymphoid organs, superinfected patient PBMC were stimulated with anti-CD3 and anti-CD28 antibodies and then cocultured with autologous PBL expanded with virus-pulsed DC with or without PI at an effector/target ratio of 1:1. Unloaded DC-treated T cells were used in parallel as a control. Cell-associated proviral DNA and supernatant viral RNA concentrations were measured by previously described quantitative assays. The proviral DNA load (copies per 106 cells) was decreased by 2 log₁₀ (P<0.001) in autologous T cells expanded with virus-pulsed DC without PI, whereas it was decreased by >3 log₁₀ (P<0.001). (i.e., below the detection threshold of 5 copies/10⁶ cells) in T cells expanded with virus-pulsed DC with PI. On the other hand, HIV RNA in the supernatants of the same cultures was decreased by 4 log₁₀ (P<0.001) and >6 log₁₀ (i.e., below the detection threshold of 10 copies/ml) in these two situations(FIGS. 4A and 4B).

Optimum suppressions of proviral DNA and supernatant RNA to levels below the detection threshold were also obtained by patient T cells stimulated with virus-pulsed DC alone when the effector/target ratio was increased to 5:1. Addition of anti-CD8 antibodies abolished these antiviral activities, while addition of anti-CD4 antibodies did not have any effect on the clearance of proviral HIV DNA or supernatant HIV RNA (FIGS. 4C and 4D). Again, the antiviral activity of autologous CD8+T cells expanded by virus-pulsed DC or by virus-pulsed DC plus PI (indinavir, 10 nM) was achieved equally in untreated and HAART-treated patients whatever their CD4 cell counts and viral load levels (P>0.3).

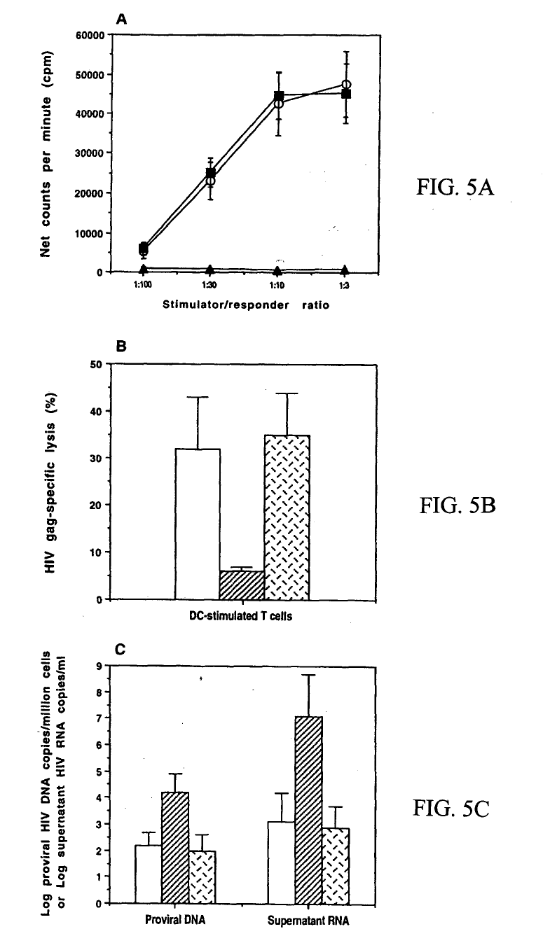

The cultures showing undetectable proviral DNA and supernatant HIV-1 RNA were further cocultured with phytohemagglutinin-stimulated normal donor PBMC for 30 days. No infectious virus was recovered from any of these cultured patient T cells that had demonstrated undetectable proviral DNA and supernatant viral RNA. DC functions following treatment with activated-T-cell-derived supernatant. Since the immune-activated lymphoid organs and tissues are the major sites for HIV replication and dissemination, we questioned whether DC could uptake and process HIV and/or present HIV antigens to effector T cells in such an immune-activated environment. Immature DC were pretreated for 2 days with the culture supernatant derived from T cells stimulated with anti-CD3/CD28 antibodies for 7 days, and then proliferation, CTL, and antiviral activities were analyzed as described above. When pretreated with activated-T cell supernatant before the virus pulse, patient DC lost their capacity to stimulate proliferation, gag-specific CTL response, and HIV-expressing cell killing of autologous T cells. However, these DC functions were preserved when the activated-T-cell supernatant was added to DC after pulsing with inactivated virus (FIG. 5). Flow cytometric analysis showed a supermaturation phenotype (up-regulated expression of CD40, CD80, CD83, CD86, and major histocompatibility complex class II) of DC following exposure to activated-T-cell supernatant.

The aforementioned above referenced data and results as described in Example 1 provide the first evidence that a high frequency of PBMC harboring HIV can be eradicated in vitro by cultured patient T cells expanded with inactivated-virus-pulsed autologous DC. This potent antiviral activity of patient T cells stimulated with virus-pulsed DC is CD8 dependent and independent of the patient's disease stage and treatment status.

Treatment of patient DC with activated-T-cell supernatant results in the loss of their integrated APC functions to present new viral antigens. These findings indicate that a disturbance in the presentation of viral antigens is most likely the cause of failure in mounting an efficient anti-HIV immunity in untreated HIV-seropositive individuals as well as in HAART treated patients despite a significant improvement of T-cell reactivity. The viral clearance obtained in vitro with autologous T cells expanded by inactivated-virus-pulsed DC opens the possibility of an in vivo restoration of anti-HIV immunity, which is readily developed in most cases shortly after infection (probably before virus dissemination into lymph notes), but is progressively lost during the course of the infection. APC functions (including up-regulation of T-cell proliferation, CTL response, and anti-HIV activities) of patient DC are enhanced by a nonantiviral concentration of PI (indinavir). This is no longer surprising, since recent in vivo and in vitro studies by our group and others show that PIs exhibit direct up-regulatory effects on proliferation and down regulatory effects on apoptosis of patient T cells following immune stimulation. Thus, a PI (at both antiviral and nonantiviral concentrations) could be used as a potent adjuvant for optimizing the virus-specific CTL response in individuals following either preventive or therapeutic vaccination.

Although the in vivo evolving HIV-1 variants that evade the antiviral immunity developed during early infection have been known for many years, the reason that the infected host fails to mount de novo mutant-virus-specific immunity remains unknown. In a chronically HIV-infected individual (i.e., one in whom the virus has already been disseminated into lymph nodes), viral replication is directly linked to local activation of lymphoid tissues characterized by huge in situ expression and release of cytokines. Certain components of these lymphoid cytokines (such as IL-10 and IFN-β) are known to interfere with generation of immature DC, and others (such as IL-1β, IL-6, TNF-α, IFN-α, IFN-γ, etc.) provoke DC maturation. Since supermatured DC lose their ability to process and present viral antigens (FIG. 4), it is conceivable that supermatured DC in immune-activated lymphoid tissues could not exert their APC function to process and present the evolving mutant antigens of viral variants. Such paralyzed DC in situ, in fact, could thus provide the prerequisite for establishing chronic HIV infection. However, our data demonstrate that such a defect in the generation of functional DC in HIV infected patients can be overcome by DC-based vaccines generated in vitro from peripheral blood monocytes taken from infected patients.

Proviral DNA of patient PBMC can disappear (or become undetectable) when cocultured with autologous T cells pretreated with virus-pulsed DC with PI, suggesting that latent forms of HIV provirus might be rare in immune-activated lymphoid tissues. Our data suggest eradicating the virus in vivo with a repeated vaccination regimen. Although HIV provirus might reside in quiescent T cells as a temporary viral reservoir escaping from recognition or killing by virus-specific effector cells, immune stimulation strategies such as IL-2-based therapy could help to activate quiescent T cells harboring HIV provirus, thereby exhausting such a temporary reservoir.

The previous example demonstrated that APC, and more specifically DCs of HIV infected patients loaded with AT-2-inactivated autologous HIV elicited functional virus-specific effector CD8+T lymphocytes which were capable of eradicating HIV-infected cells in vitro, the natural progression was to confirm duplication of such a response in vivo.

Specifically in this illustration, one aspect of the invention is described wherein the in vivo effects of AT-2-inactivated virus-pulsed DCs vaccine are demonstrated through the treatment of a SIV-infected rhesus monkey. As is well understood in the art, the genetic organization of SIV is virtually identical to HIV. SIV is 50% homologous in nucleotide sequence to HIV-1. SIV and HIV-2 exhibit close structural and immunologic properties and are 75% homologous. In view of this, and considering the close genomic relationship between humans and the rhesus monkey, it was an idea model to develop and confirm a viable vaccine for in vivo use in humans.

We demonstrated that a therapeutic vaccine made of inactivated SIV-pulsed DCs can elicit effective cellular immune responses against SIV, allowing control of SIV replication in the secondary lymphoid tissues and reduction of cell-associated viral DNA and cell-free viral RNA in blood of SIV-infected macaques. Increased circulating SMTCs associated with decreased blood viral loads observed over the first 10-24 days after the first immunization may reflect the in vivo stimulation of pre-existing memory T cells by the vaccine.

On the other hand, the higher increase in SMTCs and the deeper decrease in blood viral loads observed from day 30 to 45 may result from in vivo priming of the naive T-cell pool. This is also suggested by recent findings showing that presentation of viral antigens by infected DCs to naïve T cells in draining lymph nodes can occur as early as 6 hours inoculation. In this setting, newly primed T cells by SIV-DC vaccine may expand gradually and lead to the enrichment of virus-specific effector/memory T cells causing the sharp reduction of viral loads observed one month after the first immunization. It is likely that the loss in DCs number and function documented in chronic HIV infection may contribute to the progressive immunodeficiency associated with the chronic phase of HIV disease. Although treatment of HIV-infected patients with highly active antiretroviral therapy (HAART) regimens has led to marked reductions in HIV load and improvements in peripheral CD4+T cell counts, HAARTs do not result in the complete restoration of immune functions, and fail to mount HIV-specific immunity. We discovered that therapeutic approaches designed to generate strong HIV-specific mediated immunity using inactivated virus-pulsed DC vaccines can result in long-term immunologic control of chronic HIV disease.

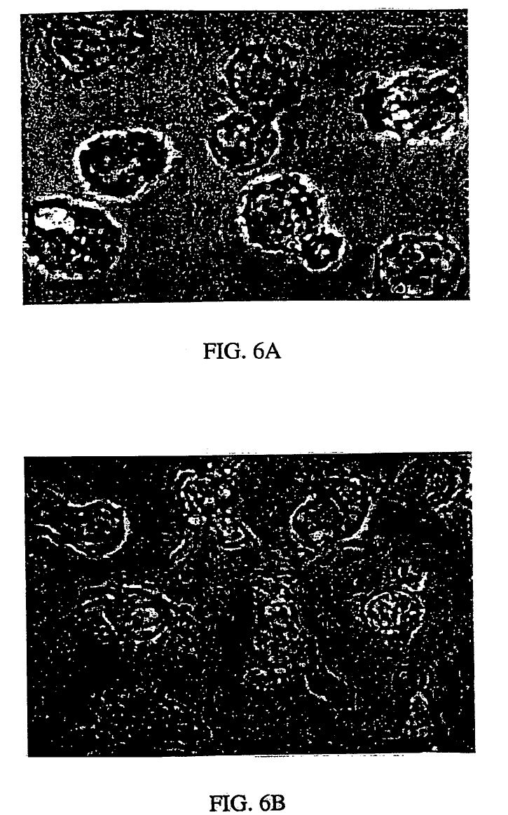

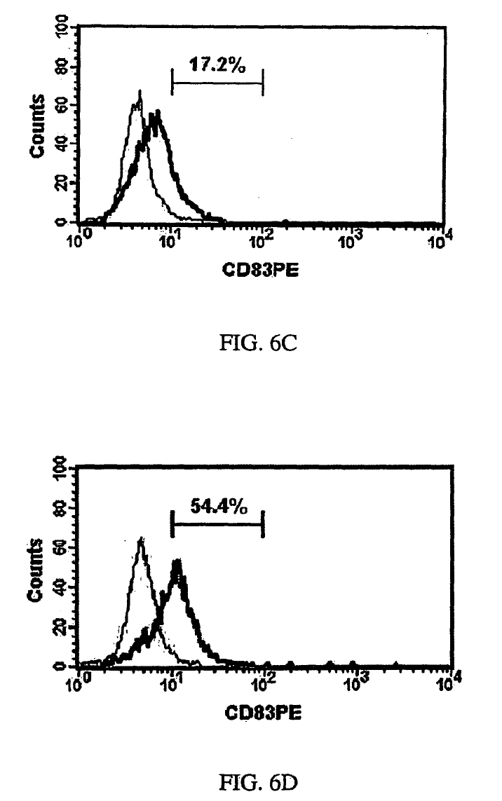

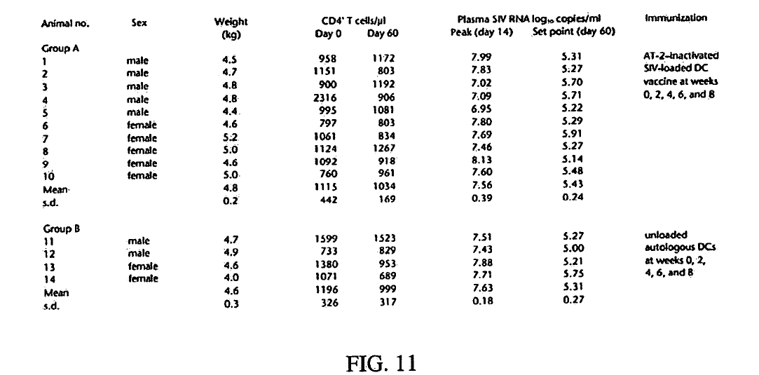

Twenty rhesus macaque monkeys (Macaca mulatta) were inoculated intravenously with five monkey infectious doses [MID1OO] of uncloned SIVmac strain 251 (SIVmac251). All animals were successfully infected. Since plasma viral RNA levels are sensitive endpoints for evaluating the efficacy of AIDS vaccines or therapies in non human primates 15, we selected the 14 (out of 20) animals who had a post infection viral load set point ranging between 105 and 106 copies/ml. Five monkeys who had a viral load set point <105 were thus excluded as well as another monkey who had a viral load set point >106 copies/ml. The 14 participating animals shared similar virologic and immunologic characteristics (FIG. 11). They were divided into two groups: group A (10 vaccinated monkeys,) and group B (4 control monkeys). Animals in group A received 4 subcutaneous immunizations (one in both forearms and thighs) with AT-2-inactivated SIVmac251-pulsed autologous DCs, whereas animals in group B received subcutaneous injections with unpulsed autologous DCs. Four booster injections with the same preparations were delivered to each animal every 2 weeks during 8 weeks. SIV-pulsed DCs demonstrated a more mature morphology and phenotype than unpulsed DCs (FIGS. 6A-6D).

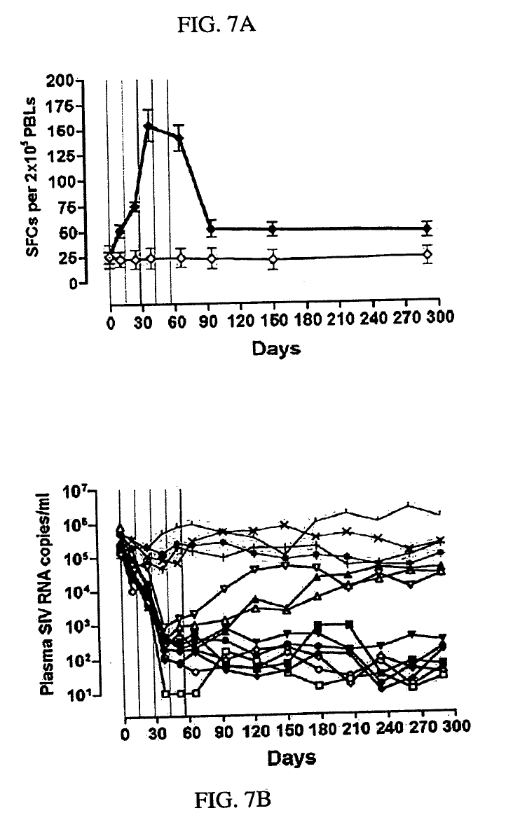

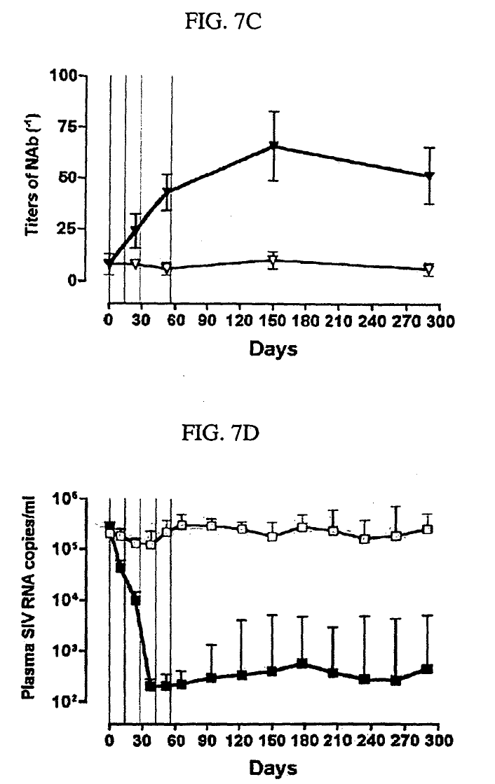

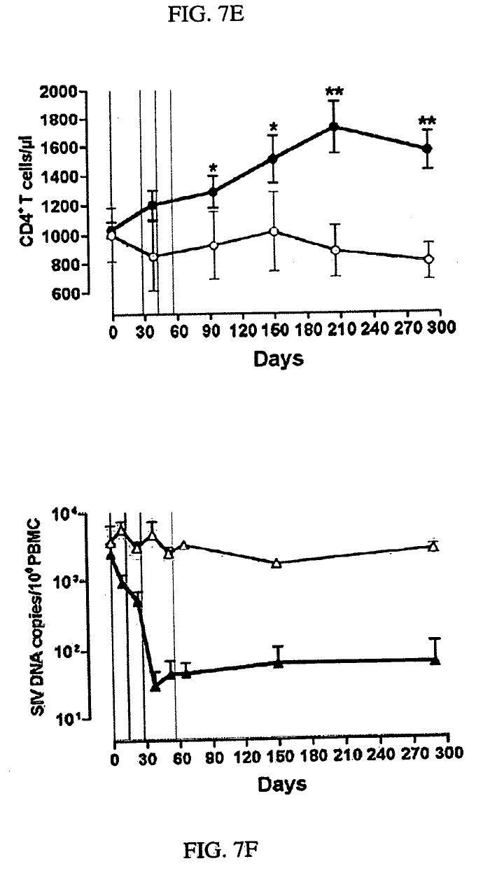

Blood SIV cellular DNA and plasmatic RNA levels (measured by quantitative PCR and RT-PCR14,16) of vaccinated monkeys (group A) started to decrease as early as 10 days after the first immunization (p<0.05). By 6 weeks (i.e. after 3 immunizations), SIV DNA and RNA levels had decreased by about 50 and 1,000 folds respectively (p<0.01). They thereafter stayed low and stable over the remaining 34 weeks (FIGS. 7A and 7B). When looking at individual vaccinated monkeys, we observed that 7 out of 10 had a well controlled viral load (<1,000 copies/ml), whereas viral loads of the remaining 3 started to re-increase (1,000 copies/mil) 17 days after the first immunization. On the other hand, SIV DNA and RNA loads of the 4 animals of the control group remained unchanged during the whole study (FIGS. 7A-7C).

In vaccinated monkeys (group A), CD4+T-cell counts increased significantly as from week 13 while they remained unchanged in the animals of groups B (FIG. 7D). No significant difference was observed in the CD4+T-cell count evolution between the seven of ten vaccinated monkeys that maintained a viral load <1,000 copies/ml and the three of ten monkeys that had a viral load >/=1000 copies/ml 8-17 weeks after immunization. Although CD8+T cells in vaccinated monkeys (but not in non-vaccinated monkeys) tended to increase, this increase remained statistically insignificant during the 40 weeks of the study. No immunodeficiency-related symptom (such as prolonged weight loss, opportunistic infections or chronic diarrhea) has been observed in the 14 animals during the test period.

Neutralizing antibody titers of the 10 vaccinated monkeys increased significantly from week 3 (P<0.05), reaching a peak that was eight-fold higher than the baseline level at week 22 (P<0.01), and still remained seven-fold higher at week 42 (P<0.01). The level of neutralizing antibodies of the three of ten vaccinated animals with a viral load >/=1000 copies/ml was significantly lower than that of the seven of ten animals with a viral load <1,000 copies/ml (P<0.01). On the other hand, neutralizing antibody titers of the four control monkeys remained low and unchanged (FIG. 7E).

Levels of circulating functional SIV-specific memory T cells (SMTC) in peripheral blood lymphocytes (PBLs) were monitored by an ELISPOT assay designed to determine the relative number of SIV antigen-specific T cells that secreted interferon-γ (IFNγ) when stimulated with SIV antigens presented by AT-2-inactivated SIV-pulsed autologous DCs. In general, all of the monkeys had a week frequency of circulating functional SMTC (mean±SE, 25±5 per 2×105 PBLs) in the baseline. Before the first vaccination, SIV-infected monkeys of both groups had a week frequency of circulating functional SMTCs (mean±SE, 25±5 per 2×105 SMTCs). In vaccinated monkeys (group A), the frequency of SMTCs increased up to about 6 fold after the third immunization (p<0.01). It then decreased, but remained, however, 2 fold above baseline level as from week 13 (p<0.05). In monkeys of the control group, SMTCs remained at their baseline levels throughout the end of the study (FIG. 7F).

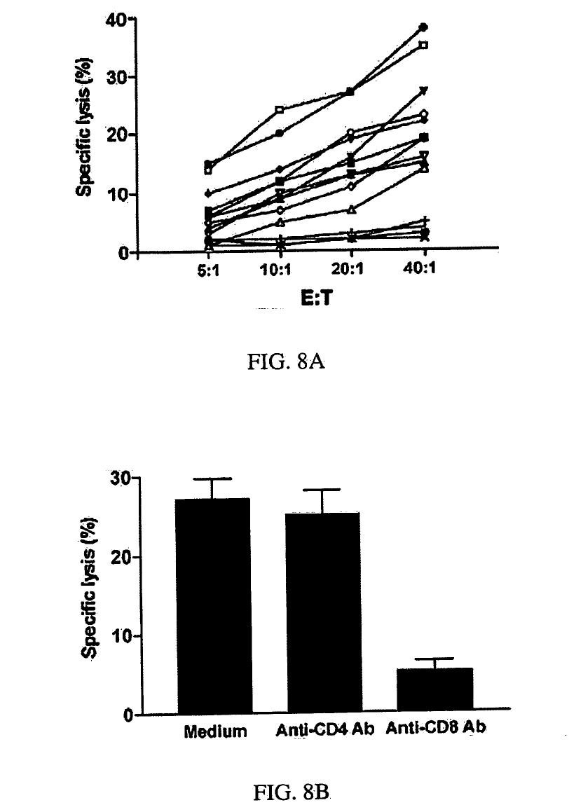

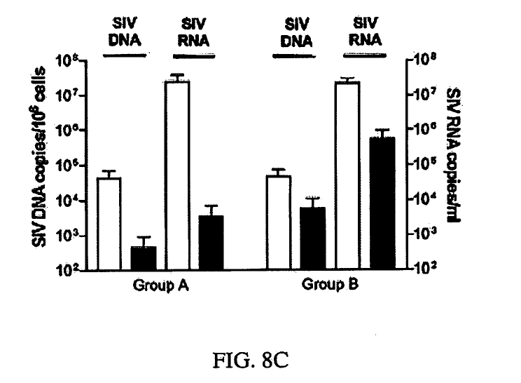

The cytolytic activity of SIV-specific effector T cells in uncultured PBLs was measured by a bulk-killing CTL assay using AT-2-inactivated SIV-pulsed autologous DCs as target of CTLs17. At week 6 (after the third vaccination), a significant increase of CTL activity was observed in all vaccinated animals (group A), but in none of the animals of the control group (Group B) at any given ratio of effector cells to target cells (P<0.01; FIG. 8A). Such CTL activity was inhibited by the addition of antibodies neutralizing CD8 (P<0.01), but was unaffected by the addition of monoclonal antibodies against CD4 (FIG. 8B). Using a previously described assay as described in Salemo-Goncalves et al., J. Virol. 74, 6648-6651 (2000)., the inhibitory activity of CD8+T cells on SIV replication autologous super-infected CD4+T cells was measured in all monkeys on PBLs taken at the 8th week. In the vaccinated monkeys (group A), SIV cellular DNA and supernatant RNA decreased by 100 and 7,000 in the presence of autologous CD8+T cells. In contrast, in the non-vaccinated animals of the control group, the decrease in SIV cellular DNA and supernatant RNA, was only 8- and 48-folds in the presence of CD8+T cells (FIG. 8C).

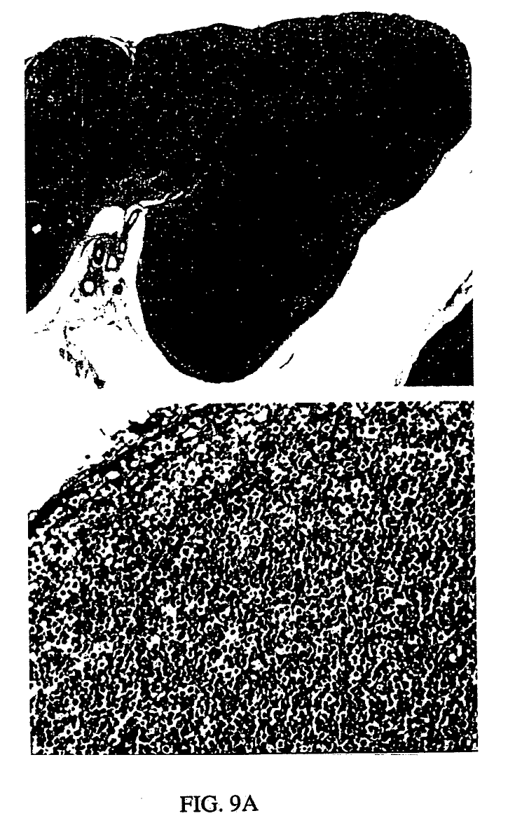

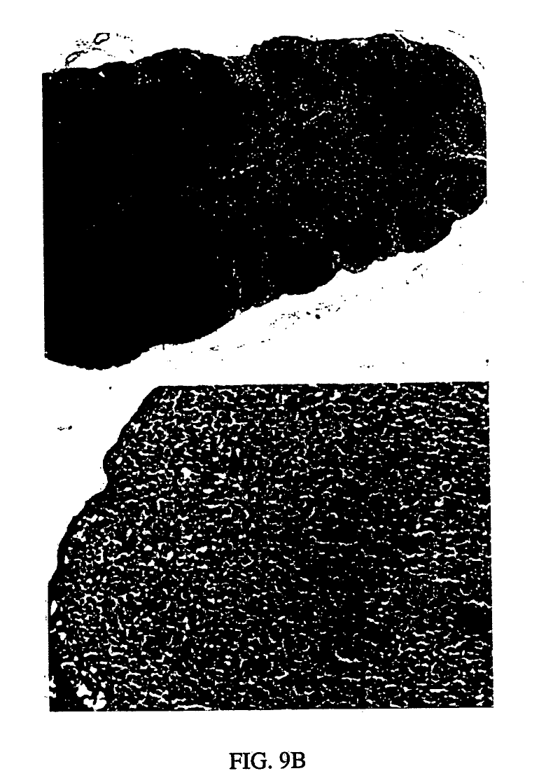

Since secondary lymphoid tissues are the major sites for SIV/HIV replication and development of virus-specific immune responses as is described in Lu, W. et al., Adv. Exp. Med. Biol. 374, 235-242 (1995); Andrieu, et al., Immunol. Today 16, 5-7 (1995), biopsies were performed in the last week to obtain axillary and inguinal lymph nodes from all animals. A typical destruction of the lymphoid follicular dendritic cell network (a histopathologic sign associated with the development of AIDS), associated with the disappearance of germinal centers, was observed in two (numbers 11 and 14) of four SIV-infected control monkeys (group B), whereas the lymphoid follicular dendritic cell network was well preserved in the ten vaccinated monkeys (group A). Representative data from monkeys 14 and 6 are shown (FIGS. 9A and 9B).

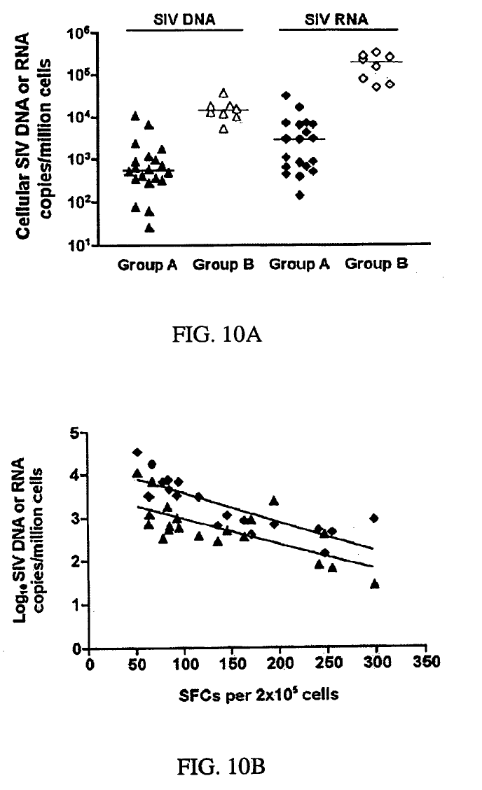

In the lymph nodes of the control group, the geometric means of the cellular SIV DNA and SIV RNA were 14,131 and 141,697 copies/million cells respectively. In contrast, in vaccinated animals (group A), these levels were 20- (p<0.01) and 70-fold (p<0.01) lower (573 and 2076 copies/million cells respectively) (FIG. 10A). SMTCs were detected in lymph nodes uncultured cells from the 20 samples of the 10 vaccinated animals (group A), but in none from the 8 samples of the 4 control animals. In vaccinated monkeys (group A), the higher was the frequency of lymph node SMTCS, the lower the SIV DNA (φ=0.507, p<0.01) and RNA burdens (φ=0.681, p<0.01) (FIG. 10B). Cytolytic- and antiviral activities of lymph nodes cells from vaccinated monkeys were further confirmed by the same bulk-killing CTL assay and functional viral activity assay as above described on PBLs (data not shown).

The results described above demonstrate that a therapeutic vaccine made of inactivated virus-pulsed DCs can elicit effective cellular and humoral immune responses against an immunodeficiency virus, allowing the control of the viral replication in the lymphoid tissues and the reduction of cell-associated viral DNA and cell-free viral RNA in blood of infected mammals. Increased circulating SMTCs associated with decreased blood viral loads observed over the first 10-24 days after the first immunization reflect the in vivo stimulation of preexisting memory T cells by the vaccine.

An effective and durable SIV-specific cellular immunity is elicited by a vaccination with chemically inactivated SIV-pulsed dendritic cells (DCs). After three immunizations made at two-week intervals, the animals exhibited a 50-fold decrease of SIV DNA and a 1,000-fold decrease of SIV RNA in peripheral blood. Such reduced viral load levels were maintained over the remaining thirty-four weeks of the study. Molecular and cellular analysis of axillary and inguinal nodes lymphocytes of vaccinated monkeys revealed the strong correlation existing between decreased SIV DNA and RNA levels and increased SIV-specific T cells responses. Inactivated whole virus-pulsed DC vaccines may be used to control immunodeficiency viruses diseases.

### REFERENCES

The subject matter of the following references, mentioned above in this specification is incorporated herein by reference:

1. Andrieu, J. M. & Lu, W. Viro-immunopathogenesis of HIV disease: implications for therapy. Immunol. Today 16, 5-7 (1995).

2. Barouch, D. H. et al. Eventual AIDS vaccine failure in a rhesus monkey by viral escape from cytotoxic T lymphocytes. Nature 415, 335-339 (2002).

3. Bohler, T., K. M. Debatin, and U. Wintergerst. 2001. T-cell apoptosis in HIV-1-infected individuals receiving highly active antiretroviral therapy. Blood 97:18981901.

4. Buseyne, F. et al. MHC-I-restricted presentation of HIV-1 virion antigens without viral replication. Nature Med. 7, 344-349 (2001).

5. Cavert, W., D. W. Notermans, K. Staskus, S. W. Wietgrefe, M. Zupancic, K. Gebhard, K. Henry, Z. Q. Zhang, R. Mills, H. McDade, C. M. Schuwirth, J. Goudsmit, S. A. Danner, and A. T. Haase. 1997. Kinetics of response in lymphoid tissues to antiretroviral therapy of HIV-1 infection. Science 276:960-964.

6. Champagne, P. et al. Skewed maturation of memory HIV-specific CD8 T lymphocytes. Nature 410, 106-111 (2001).

7. Chougnet, C. et al. Normal immune function of monocyte-derived dendritic cells from HIV-infected individuals: implications for immunotherapy. J. Immunol. 163, 1666-1673 (1999).

8. Chun, T. W., L. Stuyver, S. B. Mizell, L. A. Ehler, J. A. Mican, M. Baseler, A. L. Lloyd, M. A. Nowak, and A. S. Fauci. 1997. Presence of an inducible HIV-1 latent reservoir during highly active antiretroviral therapy. Proc. Natl. Acad. Sci. USA 94:13193-13197.

9. Cohen, O. J., Kinter, A. & Fauci, A. S. Host factors in the pathogenesis of HIV disease. Immunol. Rev. 159, 31-48 (1997).

10. Collier, A. C., R. W. Coombs, D. A. Schoenfeld, R. L. Bassett, J. Timpone, A. Baruch, M. Jones, K. Facey, C. Whitacre, V. J. McAuliffe, H. M. Friedman, T. C. Merigan, R. C. Reichman, C. Hooper, and L. Corey. 1996. Treatment of human immunodeficiency virus infection with saquinavir, zidovudine, and zalcitabine. AIDS Clinical Trials Group. N. Engl. J. Med. 334:1011-1017.

11. Davey, R. T. Jr. et al. HIV-1 and T cell dynamics after interruption of highly active antiretroviral therapy (HAART) in patients with a history of sustained viral suppression. Proc. Natl. Acad. Sci. USA 96, 15109-15114 (1999).

12. Donaghy, H. et al.Loss of blood CD11c(+) myeloid and CD11c(−) plasmacytoid dendritic cells in patients with HIV-1 infection correlates with HIV-1 RNA virus load. Blood98, 2574-2576 (2001).

13. Feldman, S. et al. Decreased interferon-production in HIV-infected patients correlates with numerical and functional deficiencies in circulating type 2 dendritic cell precursors. Clin. Immunol. 101, 201-210 (2001).

14. Fenyo, E. M. & Putkonen, P. Broad cross-neutralizing activity in serum is associated with slow progression and low risk of transmission in primate lentivirus infections. Immunol. Lett. 51, 95-99 (1996).

15. Finzi, D., J. Blankson, J. D. Siliciano, J. B. Margolick, K. Chadwick, T. Pierson, K. Smith, J. Lisziewicz, F. Lori, C. Flexner, T. C. Quinn, R. E. Chaisson, E. Rosenberg, B. Walker, S. Gange, J. Gallant, and R. F. Siliciano. 1999. Latent infection of CD4+T cells provides a mechanism for lifelong persistence of HIV-1, even in patients on effective combination therapy. Nat. Med. 5:512-517.

16. Grabar, S. et al. Clinical outcome of patients with HIV-1 infection according to immunologic and virologic response after 6 months of highly active antiretroviral therapy. Ann. Intern. Med. 133, 401-410 (2000).

17. Grassi, F. et al. Depletion in blood CD11c-positive dendritic cells from HIV-infected patients. Aids 13, 759-766 (1999).

18. Gray, C. M. et al.Frequency of class I HLA-restricted anti-HIV CD8+T cells in individuals receiving highly active antiretroviral therapy (HAART). J. Immunol. 162, 1780-1788 (1999).

19. Harrigan, P. R., M. Whaley, and J. S. Montaner. 1999. Rate of HIV-1 RNA rebound upon stopping antiretroviral therapy. AIDS 13:F59-F62.

20. Hatano, H. et al. Pre-HAART HIV burden approximates post-HAART viral levels following interruption of therapy in patients with sustained viral suppression. Aids 14, 1357-1363 (2000).

21. Haynes, B. F., G. Pantaleo, and A. S. Fauci. 1996. Toward an understanding of the correlates of protective immunity to HIV infection. Science 271:324-328.

22. Hermans, I. F., Ritchie, D. S., Yang, J., Roberts, J. M. & Ronchese, F. CD8+T cell-dependent elimination of dendritic cells in vivo limits the induction of antitumor immunity. J. Immunol. 164, 3095-3101 (2000).

23. Hirsch, V. M. & Lifson, J. D. Simian immunodeficiency virus infection of monkeys as a model system for the study of AIDS pathogenesis, treatment, and prevention. Adv. Pharmacol. 49, 437-477 (2000).

24. Ibanez, A., T. Puig, J. Elias, B. Clotet, L. Ruiz, and M. A. Martinez. 1999. Quantification of integrated and total HIV-1 DNA after long-term highly active antiretroviral therapy in HIV-1-infected patients. AIDS 13:1045-1049.

25. Jin, X. et al. An antigenic threshold for maintaining human immunodeficiency virus type 1-specific cytotoxic T lymphocytes. Mol. Med. 6, 803-809 (2000).

26. Kalams, S. A. et al. Levels of human immunodeficiency virus type 1-specific cytotoxic T-lymphocyte effector and memory responses decline after suppression of viremia with highly active antiretroviral therapy. J. Virol. 73, 6721-6728 (1999).

27. Kaufmann, D., G. Pantaleo, P. Sudre, and A. Telenti. 1998. CD4-cell count in HIV-1-infected individuals remaining viraemic with highly active antiretroviral therapy (HAART). Swiss HIV Cohort Study. Lancet 351:723-724.

28. Knight, S. C. & Stagg, A. J. Antigen-presenting cell types. Curr. Opin. Immunol. 5, 374-382 (1993).

29. Kostense, S. et al. Persistent numbers of tetramer(+) CD8(+) T cells, but loss of interferon-(+) HIV-specific T cells during progression to AIDS. Blood 99, 25052511 (2002).

30. Ledergerber, B., M. Egger, M. Opravil, A. Telenti, B. Hirschel, M. Battegay, P. Vernazza, P. Sudre, M. Flepp, H. Furrer, P. Francioli, and R. Weber. 1999. Clinical progression and virological failure on highly active antiretroviral therapy in HIV-1 patients: a prospective cohort study. Swiss HIV Cohort Study. Lancet 353:863-868.

31. Lederman, M. M. & Valdez, H. Immune restoration with antiretroviral therapies: implications for clinical management. JAMA284, 223-228 (2000).

32. Levitz, S. M. 1998. Improvement in CD4+cell counts despite persistently detectable HIV load. N. Engl. J. Med. 338:1074-1075.

33. Lieberman, J., Shankar, P., Manjunath, N. & Andersson, J. Dressed to kill? A review of why antiviral CD8 T lymphocytes fail to prevent progressive immunodeficiency in HIV-1 infection. Blood 98, 1667-1677 (2001).

34. Lu, W. & Andrieu, J. M. HIV protease inhibitors restore impaired T-cell proliferative response in vivo and in vitro: a viral-suppression-independent mechanism. Blood 96, 250-258 (2000).

35. Lu, W. & Andrieu, J. M. In vitro HIV eradication by autologous CD8+T cells expanded with inactivated-virus-pulsed dendritic cells. J. Virol. 75, 8949-8956 (2001).

36. Lu, W. & Andrieu, J. M. Prospective views of HIV pathology. Clues for therapeutic strategies. Adv. Exp. Med. Biol. 374, 235-242 (1995).

37. Lu, W., Achour, A., Arlie, M., Cao, L. & Andrieu, J. M. Enhanced dendritic-cells-driven proliferation and anti-HIV activity of CD8+T cells by a new phenothiazine derivative aminoperazine. J. Immunol. 167, 2929-2935 (2001).

38. Lu, W., and J. M. Andrieu. 1995. Prospective views of HIV pathology. Clues for therapeutic strategies. Adv. Exp. Med. Biol. 374:235-242.

39. Lu, W., and J. M. Andrieu. 2000. HIV protease inhibitors restore impaired T-cell proliferative response in vivo and in vitro: a viral-suppression-independent mechanism. Blood 96:250-258.

40. Lu, W., and J. M. Andrieu. 2001. T-cell recovery in HIV-infected patients experiencing virologic failure under highly active antiretroviral therapy. Blood 97:1900-1901.

41. Lu, W., D. S. Han, J. Yuan, and J. M. Andrieu. 1994. Multi-target PCR analysis by capillary electrophoresis and laser-induced fluorescence. Nature 368:269-271.

42. Lu, W., L. Cao, L. Ty, M. Arlie, and J. M. Andrieu. 1999. Equivalent amplification of intrinsically variable nucleic acid sequences by multiple-primer-induced overlapping amplification assay: applications for universal detection and quantitation. Nat. Med. 5:1081-1085.

43. Ludewig, B. et al. Protective antiviral cytotoxic T cell memory is most efficiently maintained by restimulation via dendritic cells. J. Immunol. 163, 1839-1844 (1999).

44. McIlroy, D. et al.Low CD83, but normal MHC class II and costimulatory molecule expression, on spleen dendritic cells from HIV+patients. AIDS Res. Hum. Retroviruses 14, 505-513 (1998).

45. McMichael, A. J., and R. E. Phillips. 1997. Escape of human immunodeficiency virus from immune control. Annu. Rev. Immunol. 15:271-296.

46. Mehlhop, E. et al. Enhanced in vitro stimulation of rhesus macaque dendritic cells for activation of SIV-specific T cell responses. J. Immunol. Methods 260, 19-234 (2002).

47. Nair, S. K. et al. Induction of primary carcinoembryonic antigen (CEA)-specific cytotoxic T lymphocytes in vitro using human dendritic cells transfected with RNA. Nature Biotechnol. 16, 364-369 (1998).

48. Norbury, C. C., Malide, D., Gibbs, J. S., Bennink, J. R. & Yewdell, J. W. Visualizing priming of virus-specific CD8+T cells by infected dendritic cells in vivo. Nature Immunol. 3, 265-271 (2002).

49. Pacanowski, J. et al. Reduced blood CD123+(lymphoid) and CD11c+(myeloid) dendritic cell numbers in primary HIV-1 infection. Blood 98, 3016-3021 (2001).

50. Rossio, J. L., M. T. Esser, K. Suryanarayana, D. K. Schneider, J. W. Bess, Jr., G. M. Vasquez, T. A. Wiltrout, E. Chertova, M. K. Grimes, Q. Sattentau, L. O. Arthur, L. E. Henderson, and J. D. Lifson. 1998. Inactivation of human immunodeficiency virus type 1 infectivity with preservation of conformational and functional integrity of virion surface proteins. J. Virol. 72:7992-8001.

51. Rossio, J. L. et al. Inactivation of human immunodeficiency virus type 1 infectivity with preservation of conformational and functional integrity of virion surface proteins. J. Virol. 72, 7992-8001 (1998).

52. Salerno-Goncalves, R., Lu, W. & Andrieu, J. M. Quantitative analysis of the antiviral activity of CD8(+) T cells from human immunodeficiency virus-positive asymptomatic patients with different rates of CD4(+) T-cell decrease. J. Virol. 74, 6648-6651 (2000).

53. Salerno-Goncalves, R., W. Lu, A. Achour, and J. M. Andrieu. 1998. Lysis of CD4+T cells expressing HIV-1 gag peptides by gag-specific CD8+cytotoxic T cells. Immunol. Lett. 64:71-77.

54. Santini, S. M., C. Lapenta, M. Logozzi, S. Parlato, M. Spada, T. Di Pucchio, and F. Belardelli. 2000. Type I interferon as a powerful adjuvant for monocyte-derived dendritic cell development and activity in vitro and in Hu-PBL-SCID mice. J. Exp. Med. 191:1777-1788.

55. Sapp, M. et al. Dendritic cells generated from blood monocytes of HIV-1 patients are not infected and act as competent antigen presenting cells eliciting potent T-cell responses. Immunol. Lett. 66, 121-128 (1999).

56. Sloand, E. M., P. N. Kumar, S. Kim, A. Chaudhuri, F. F. Weichold, and N. S. Young. 1999. Human immunodeficiency virus type 1 protease inhibitor modulates activation of peripheral blood CD4(+) T cells and decreases their susceptibility to apoptosis in vitro and in vivo. Blood 94:1021-1027.

57. Wilson, C. C., W. C. Olson, T. Tuting, C. R. Rinaldo, M. T. Lotze, and W. J. Storkus. 1999. HIV-1-specific CTL responses primed in vitro by blood-derived dendritic cells and Th1-biasing cytokines. J. Immunol. 162:3070-3078.

58. Zhang, Z., T. Schuler, M. Zupancic, S. Wietgrefe, K. A. Staskus, K. A. Reimann, T. A. Reinhart, M. Rogan, W. Cavert, C. J. Miller, R. S. Veazey, D. Notermans, S. Little, S. A. Danner, D. D. Richman, D. Havlir, J. Wong, H. L. Jordan, T. W. Schacker, P. Racz, K. Tenner-Racz, N. L. Letvin, S. Wolinsky, and A. T. Haase. 1999. Sexual transmission and propagation of SIV and HIV in resting and activated CD4+T cells. Science 286:1353-1357.

59. Zimmer, M. I. et al. Disrupted homeostasis of Langerhans cells and interdigitating dendritic cells in monkeys with AIDS. Blood 99, 2859-2868 (2002).

What is claimed is:

1. A composition for initiating an immune response against an immunodeficiency virus comprising an antigen-presenting cell pulsed with an inactivated virus.

2. The composition of claim 1, wherein the composition initiates an immune response against a Human Immunodeficiency Virus (HIV).

3. The composition of claim 1, wherein the composition initiates an immune response against a Simian Immunodeficiency Virus (SIV).

4. The composition of claim 1, wherein the antigen presenting cell is a monocyte-derived dendritic cell.

5. The composition of claim 4, wherein the monocyte-derived dendritic cell is immature.

6. The composition of claim 1 further comprising a protease inhibitor.

7. The composition of claim 6, wherein the protease inhibitor is an Human Immunodeficiency Virus (HIV) protease inhibitor.

8. The composition of claim 7, wherein said HIV protease inhibitor is indinavir.

9. The composition of claim 6, wherein said HIV protease inhibitor is present in the composition at non-antiviral doses.

10. The composition of claim 6, wherein said HIV protease inhibitor is present in the composition at antiviral doses.

11. The composition of claim 1, wherein said inactivated-virus is an inactivated autologous virus.

12. The composition of claim 11, wherein said inactivated-autologous-virus is an inactivated autologous human immunodeficiency virus (HIV).

13. The composition of claim 11, wherein said inactivated-autologous-virus is an inactivated autologous simian immunodeficiency virus (SIV).

14. A composition for expanding expression of virus-specific CB8+T cells, comprising an autologous dendritic cell pulsed with an inactivated human immunodeficiency virus (HIV).

15. The composition of claim 14, wherein said virus-specific CB8+T cells kill HIV-infected cells.

16. The composition of claim 14, wherein said virus-specific CB8+T cells suppress HIV Type 1 (HIV-1) replication.

17. The composition of claim 14, wherein said virus-specific CB8+T cells substantially eradicate HIV-1 in peripheral blood mononuclear cells (PBMC).

18. The composition of claim 14, which increases HIV-specific cytotoxic-T-lymphocyte (CTLE) activity of autologus peripheral blood lymphocytes.

19. A method of eradicating HIV infected cells in a mammal comprising:
culturing T cells from the mammal;
expanding said T cells with an inactivated-virus-pulsed autologous dendritic cell; and
exposing said cells harboring HIV to the T cells expanded with the inactivated-virus-pulsed autologus dendritic cell.

20. A composition for initiating an immune response against human immunodeficiency virus (HIV) comprising an inactivated human immunodeficiency virus (HIV)-pulsed dendritic cell, wherein said inactivated-HIV-pulsed dendritic cell expands expression of virus-specific CD8+T cells which kill HIV-infected cells.

21. A method of controlling an immunodeficiency viral load of a mammal, comprising the steps of administering the composition of claim 1 at a dosage and for a time sufficient to reduce the immunodeficiency viral load.

22. A method of inducing an immune response in a mammal, comprising administering the composition according to claim 1 to said mammal at a dosage and for a time sufficient to induce protective immunity against subsequent infection.

23. A method of inducing production of antibodies to HIV in a human, comprising administering to the human a substantially purified dendritic cell pulsed with an inactivated HIV.

24. A method of inducing production of antibodies to SIV in a monkey, comprising administering to the monkey substantially purified and inactivated SW-pulsed dendritic cells.

25. An anti-HIV vaccine comprising autologous inactivated whole HIV-pulsed dendritic cells.

26. A method of inducing an anti-HIV immune response in a mammal, comprising administering the vaccine of claim 25 to said mammal in a therapeutically effective dosage effective to elicit an immune response to protect said mammal against subsequent infection with at least one strain of HIV.

27. A method of inducing production of anti-HIV immunity in a mammal comprising administering to the mammal:
a substantially purified autologous HIV-virus; and/or
an autologous host cell infected with the inactivated autologous HIV-virus; and
an autologous host cell infected with an autologous attenuated HIV-virus.

28. The method of claim 27, further comprising recovering and purifying said anti-HIV antibodies.

29. A method of treating a mammal for HIV infection, comprising administering an antibody obtained by the method of claim 27 to said mammal in a therapeutically effective dosage to reduce one or more symptoms associated with said HIV infection.

30. A composition for expanding the expression of virus-specific CD8+T cells which kill HIV-infected cells and suppressing HIV type 1 (HIV-1) replication, comprising an inactivated virus pulsed dendritic cell and an HIV protease inhibitor.

31. A method of inducing production of anti-HIV immunity in a mammal comprising administering to the mammal an autologous dendritic cell pulsed with the inactivated autologous HIV-virus.

32. The method of claim 31, further comprising the step of administering an HIV protease inhibitor.
